(19) United States
(12) Patent Application Publication
(10) Pub. No.: US 2001/0047210 A1
(43) Pub. Date: Nov. 29, 2001

(54) Facile total shoulder arthroplasty apparatus and method

(76) Inventor: Eugene M. Wolf, San Rafael, CA (US)

Correspondence Address: Andrew V. Smith, Sierra Patent Group, Ltd., P.O. Box 6149, Stateline, NV, 89449, US

(21) Appl. No.: 09/811,360

(22) Filed: Mar. 16, 2001

Related U.S. Application Data
(60) Provisional application No. 60/189,752, filed on Mar. 16, 2000.

(51) Int. Cl. A61F 2/40

(52) U.S. Cl. 623/19.14; 606/87; 606/99

(57) ABSTRACT

A shoulder arthroplasty apparatus includes a stemless humeral head for coupling to a previously cut humeral surface. The base of the humeral head has a stabilizing base extension that is impacted into a cancellous region of the cut humeral surface. A preferred base extension has a cruciform shape including two planar fins intersecting at or near the center of the base. The periphery of the humeral head is formed to match the cortical margins of the cut humeral surface. The humeral head is preferably cemented or press-fit to the humeral surface. The humeral head has any of a range of peripheral shapes and sizes matching the specific shape and size of the anatomic neck of a humeral surface. A method for performing the shoulder arthroplasty includes a new surgical approach using a guide to cut accurately and safely on the anatomic neck of the humerus and another guide to properly orient the reaming of the glenoid.

### PRIORITY

This application claims the benefit of priority to U.S. provisional patent application no. 60/189,752, filed Mar. 16, 2000.

### BACKGROUND OF THE INVENTION

1. Field of the Invention

The invention relates to shoulder arthroplasty, and particularly to total shoulder arthroplasty using a anatomic humeral component that is load bearing on the cut humeral surface with a base extension to resist rotational forces. This humeral head prosthesis will recreate an anatomic proximal humeral configuration without the need to conform and fixate to an intramedullary stem.

2. Discussion of the Related Art

Most practicing orthopedic surgeons are uncomfortable performing total shoulder arthroplasty (TSA). Statistically the general orthopedic surgeon does less than one total shoulder arthroplasty per year. The existing techniques are demanding and the complication rate relatively high when compared to total knee or total hip arthroplasty. Orthopedic surgeons in general orthopedic practice are inexperienced in total shoulder arthroplasty, and often refer them to shoulder specialists when a shoulder arthroplasty is necessary.

Many primary care physicians do not even know that a prosthetic total shoulder arthroplasty exists as an option for their patients with severe glenohumeral arthritis. These physicians will also tend to treat patients with severe glenohumeral arthritis with medicinal therapies for an extensive period of time and defer referrals to orthopedic surgeons.

### RECOGNIZED IN THE INVENTION

The total number of shoulder prosthetic replacements performed are constrained by the above factors. It is desired to improve the methods for performing TSA and improve the perceptions of the procedure in the orthopedic as well as the general medical community. Some improvements that can be made that may help to achieve this include:

greatly facilitating the operative technique

offer special hands-on training to the orthopedic surgeons

decreasing the post-operative morbidity

accelerating the rehabilitation

improving the short and long term results

reducing the cost of TSA

It ie recognized in the present invention that a TSA system solution should have some or all of the following features to efficiently and effectively create the ideal TSA:

It is desired to efficiently and effectively recreate an anatomic proximal humeral configuration. Since the introduction of the Aequalis shoulder prosthesis (Tournier S.A., Saint-Ismier, France, see FIGS. 1a-1b) the notion of recreating the postero-medial offset and head-shaft angle has become the new standard for shoulder prostheses (see Boileau et al., above). But the technique of the Tournier product is difficult and lacking in instrumentation that would make the operation accessible to a greater number of orthopedic surgeons. The Aequalis TSA technique as well as most recent shoulder prosthetic systems are demanding and complex. The humeral prostheses are modular and can have multiple components and are very expensive.

It is desired to perform a TSA efficiently. Medicare's DRG (491) reimbursement is a fixed amount and for 1998 that amount was $7,089 for the entirety of the total shoulder event. At this reimbursement, it is difficult for hospitals in the US to provide an anatomic prosthesis without sustaining a considerable loss in every case covered by Medicare (63.8% of such cases are covered by Medicare). HMO contracts for TSA are even worse and are usually a negotiated rate that is less than Medicare.

Existing TSA designs are simply too complex and expensive. The present state of reimbursement of TSA makes it a losing financial event for providers in most cases. It is desired, then, to have a TSA apparatus and technique that permits recreation of a normal anatomic proximal humeral configuration in a simple and cost-effective way.

It is desired to provide an accurate cutting guide for the anatomic neck of the proximal humerus. The anatomic systems do not provide a cutting guide for the proximal humerus. There is considerable variability of retroversion of the humeral head (see Boileau et al., and Pearl et al., cited above). Other existing TSA systems cut the humerus to match the inclination of the prosthesis at an arbitrary angle of retroversion. It is however desired that the proximal humeral cut match the anatomic neck of the patient's humerus. In addition, the guide should provide protection of the rotator cuff. The rotator cuff can be seriously damaged if the proximal humeral cut extends into the greater tuberosity where the cuff attaches. A total shoulder arthroplasty with a deficient rotator cuff will produce poor results, complications, and simply fail.

It is desired to provide an accurate guide to ensure that the glenoid is reamed in a plane that is perpendicular to the axis of the scapula. There is no guide presently available to assist in placement of the glenoid in proper version with respect to the scapula. This is of particular interest in cases where there is the need to address the problem of excessive posterior glenoid wear. There is no guide available in any total shoulder system to guide the corrective reaming that is necessary in these cases. Asking the surgeon to “eyeball” this correction is unacceptable because once a reamer is placed in the open shoulder in contact with the glenoid, the glenoid is no longer in view. The reamer is then removed repeatedly after short bursts of reaming to assess the amount of bone removed, as well as the area of the glenoid that the reaming is affecting.

It is desired to facilitate the difficult access to the glenoid by improved retractors and a better surgical approach. The glenoid preparation and component insertion is one of the most technically difficult aspects of total shoulder operations. Access to the glenoid involves extensive dissection that risks the axillary nerve as well as more major neurovascular structures. This extensive dissection is performed due to deficiencies in present glenoid component designs and the instrumentation necessary to prepare the glenoid. Even under the best conditions, glenoid loosening remains a concern and is estimated to occur at one percent per year. The difficulty in glenoid preparation and insertion is so substantial that even the most experienced shoulder specialists have omitted the glenoid component and performed hemiarthroplasties for glenohumeral arthritis (see Boileau, et al., mentioned above). Of the reports of complications of total shoulder arthroplasty, neurological loss due to axillary nerve damage and more severe diffuse brachial plexus injuries are not uncommon. These injuries are due to the need to release the capsule in the area of the axillary nerve and to the vigorous posterior retraction necessary to access the glenoid and humeral shaft.

It is desired to reduce the risk of post-operative instability of TSA components. The risk of occurrence of post-operative instabilities can be reduced if the extent of capsulo-ligamentous dissection and release can be kept to a minimum. The designers of the Aequalis and other TSA systems recommend a complete anterior and posterior capsular release to be able to access the glenoid and use their glenoid preparation instrumentation. This can produce considerable posterior instability that requires multiple “suspension” or salvage sutures from the posterior capsule to anterior structures like the subscapularis and/or to the conjoined tendon. The difficult glenoid access produces the need for these extensive capsular releases. It is therefore desired to have reduce or avoid such complexities arising from the difficult glenoid access.

It is desired to facilitate the revision of a TSA. Revision of a TSA is the most daunting of all revision arthroplasties. The removal of a cemented or an ingrowth stem from the fragile proximal humerus is difficult and is often accomplished by bivalving the entire length of the humerus where cement or ingrowth may exist. This requires an extensive exposure of the proximal half of the humerus, and produces unacceptable morbidity and potential for complications. It is desired to have a TSA apparatus and technique wherein revisions may be simply performed without such unacceptable morbidity.

### SUMMARY OF THE INVENTION

It is therefore an object of the invention to have an effective and efficient shoulder arthroplasty that permits recreation of a normal anatomic proximal humeral configuration with a surface bearing humeral component as opposed to stem bearing humeral components now in use.

In accord with the above object, a total shoulder arthroplasty apparatus includes a stemless humeral head for coupling to a previously cut humeral surface. The base of the humeral head has a stabilizing base extension that is impacted into a cancellous region of the cut humeral surface.

Preferably the humeral head is configured such that the base extension protrudes only into a ball region of the humerus of the patient, and not into an elongate region of the humerus. In this sense, it is preferred that the base extension be formed such that the humeral head is nonintrusive of the elongate region of the humerus when the humeral head is impacted to the cancellous interior of the cut humerus.

The protruding base extension may include one or more planar fins or extensions, or two or more linear extensions, or a combination of planar and linear extensions, for efficient rotational stabilization. A preferred base extension has a cruciform shape wherein two planar extensions intersect at or neat the center of the base. The periphery of the humeral head matches the cortical margins of the cut humeral surface.

The humeral head is preferably attached to the humeral surface using a methylmethacrylate cement. Alternatively, the humeral head's contact surface is porous and is press-fit to allow ingrowth from the humeral surface. The humeral head may have any of a range base diameters, preferably between 38 to 56 mm, and may be asymmetrically shaped although the base is preferably substantially circular, matching the anatomic neck of a humeral surface. In one embodiment, the base extension is impacted into the cut humeral surface where a template punch has been previously inserted into the humeral surface, wherein the extension is of the same size as the template punch.

Further in accord with the above object, a method for total shoulder arthroplasty includes preparing a humeral head having a stabilizing extension from the base for rotational stabilization of the humeral head on a cut humeral surface for coupling to the humeral surface. The method then includes preparing a humeral surface for fixation of the humeral head thereon, including cutting the humeral surface to reveal a cancellous interior, and coupling the humeral head to the humeral surface, preferably by impacting the inferior extension into the cancellous of the cut humeral surface, wherein the periphery of the humeral head rests on the cortical margins of the humeral surface following the attaching.

Preferably, the coupling step includes cementing the humeral head to the humeral surface, and alternatively, the coupling step includes press-fitting the humeral head to the humeral surface. The humeral head forming step preferably also includes selecting a peripheral shape and size from a variety of shapes and sizes for fitting to the specific design of the anatomic neck of the humeral surface. In one embodiment, a step of inserting a template punch into the cancellous of the cut humeral surface prior to performing the impacting step is preferred, wherein the inferior extension is a total or partial male complement to the female template punch.

In another aspect of the invention, the surgical approach is a supero-lateral deltoid splitting approach as opposed to the delto-pectoral approach used in all other systems. This approach will allow much improved access to the glenoid. The skin incision is from the postero-lateral corner of the acromion to the axillary crease. After subcutaneous undermining the deep layer incision begins on the superior surface of the clavicle, continues over the acromioclavicular joint and anterior margin of the acromion. The deltoid is the split to the extent of the subacromial bursa. The axillary nerve is palpated and protected during the procedure. The acromio-clavicular capsule and coraco-acromial ligament is dissected from their bony attachments and tagged to insure their restoration on closing. Three centimeters of anterior deltoid medial to the acromio-clavicular joint is dissected subperiosteally from the clavicle. This will create a large anterolateral access to the glenohumeral joint. The glenohumeral joint is then opened in the usual fashion by cutting through the subscapularis and capsule. This approach avoids easier and direct glenoid access without posterior capsular releases and vigorous retraction.

The method includes a cutting jig to guide an oscillating saw to cut at the anatomic neck of the humerus. There will be right and left shoulder guide for this purpose. The guide will be similar to a large tenaculum and will open and close onto the anatomic neck. It will have a variable axis as a typical pair of pliers. It will preferably encompass only 240 degrees of the circumference of the anatomical neck. The guide will protect the rotator cuff and make use of the bald non-articular area posteriorly using a preferred 8 mm thickness posteriorly to correctly cut on the anatomic neck. The glenoid version guide is another advantageous tool in cases of excessive posterior wear or any type of glenoid destruction that creates alignment (version) problems.

In further view of the above object, a total shoulder arthroplasty apparatus for recreating an anatomic proximal humeral configuration is provided including a stemless humeral head for coupling to a previously cut humeral surface, wherein the humeral head includes a base having a non stem-bearing stabilizing base extension protruding therefrom for impaction into a cancellous region of the cut humeral surface.

In further view of the above object, a total shoulder arthroplasty apparatus for recreating an anatomic proximal humeral configuration is provided including a stemless humeral head for coupling to a cut humeral surface, wherein the humeral head includes a base having a stabilizing base extension protruding therefrom for impaction into a cancellous, nonintramedullary region of the cut humeral surface.

In further view of the above object, a total shoulder arthroplasty apparatus for recreating an anatomic proximal humeral configuration is provided including a humeral head for coupling to a cut humeral surface, wherein the humeral head includes a base having a stabilizing base extension protruding therefrom for impaction into a cancellous region of the cut humeral surface, and wherein the base extension is confined to protrude only into a ball region of the humerus, to which the humeral head couples, and which is above an elongate region of the humerus.

In further view of the above object, a total shoulder arthroplasty apparatus for recreating an anatomic proximal humeral configuration is provided including a humeral head for coupling to a cut humeral surface, wherein the humeral head includes a base having a stabilizing base extension protruding therefrom for impaction into a cancellous region of the cut humeral surface, and wherein the extension is nonintrusive of an elongate humeral region below a humeral ball region including the humeral head.

In further view of the above object, a total shoulder arthroplasty method for recreating an anatomic proximal humeral configuration is provided including preparing a stemless humeral head having a base including a stabilizing base extension for efficient rotational stabilization of the humeral head on a cut humeral surface for coupling with the cut humeral surface, preparing a humeral surface for coupling the humeral head thereto, including cutting the humeral surface to reveal a cancellous interior, and coupling the humeral head to the humeral surface, thereby recreating the anatomic proximal humeral configuration.

In further view of the above object, a total shoulder arthroplasty method for recreating an anatomic proximal humeral configuration is provided including preparing a stemless humeral head having a base including a non stem-bearing stabilizing base extension for rotational stabilization of the humeral head on a cut humeral surface for coupling to the cut humeral surface, preparing a humeral surface for coupling the humeral head thereto, including cutting the humeral surface to reveal a cancellous interior, and coupling the humeral head to the humeral surface, thereby recreating the anatomic proximal humeral configuration.

In further view of the above, a total shoulder arthroplasty method for recreating an anatomic proximal humeral configuration is provided, including preparing a stemless humeral head having a base including a non stem-bearing stabilizing base extension for rotational stabilization of the humeral head on a cut humeral surface for coupling to the cut humeral surface, preparing a humeral surface for coupling the humeral head thereto, including cutting the humeral surface to reveal a cancellous interior, and coupling the humeral head to the humeral surface, thereby recreating the anatomic proximal humeral configuration, including impacting the base extension of the humeral head to protrude only into a ball region of the humerus above an elongate region of the humerus.

In further view of the above, a total shoulder arthroplasty method for recreating an anatomic proximal humeral configuration is provided including preparing a stemless humeral head having a base including a non stem-bearing stabilizing base extension for rotational stabilization of the humeral head on a cut humeral surface for coupling to the cut humeral surface, preparing a humeral surface for coupling the humeral head thereto, including cutting the humeral surface to reveal a cancellous interior, and coupling the humeral head to the humeral surface, thereby recreating the anatomic proximal humeral configuration, including impacting the base extension of the humeral head nonintrusive to an elongate region of the humerus below a ball region of the humerus.

In further view of the above, a method for performing a shoulder arthroplasty is provided including surgically establishing an access to a humerus of a patient, coupling a guide to the humerus, wherein the humeral head remains exposed, positioning said guide to define a humeral surface, and removing said humeral head by cutting along said humeral surface defined by said guide, whereby a precise humeral surface is revealed for attaching an artifical humeral head during said arthroplasty.

In further view of the above, a total shoulder arthroplasty method for recreating an anatomic proximal humeral configuration is provided including preparing a stemless humeral head having a base including a non stem-bearing stabilizing base extension for rotational stabilization of the humeral head on a cut humeral surface for coupling to the cut humeral surface, preparing a humeral surface for coupling the humeral head thereto, including cutting the humeral surface to reveal a cancellous interior, and coupling the humeral head to the humeral surface, thereby recreating the anatomic proximal humeral configuration.

### BRIEF DESCRIPTION OF THE DRAWINGS

FIGS. 1a-1b illustrate the conventional Aequalis/Tournier prosthesis.

FIG. 2a schematically shows a side view of a humeral head for recreating an anatomical proximate humeral configuration in a shoulder arthroplasty in accord with a preferred embodiment.

FIG. 2b schematically shows a bottom view of the humeral head of FIG. 2a.

FIG. 3a schematically shows a side view of a humeral head for recreating an anatomical proximate humeral configuration in a shoulder arthroplasty in accord with a first alternative embodiment.

FIG. 3b schematically shows a bottom view of the humeral head of FIG. 3a.

FIG. 3c schematically shows an alternative bottom view of the humeral head of FIG. 3a.

FIG. 4a schematically shows a side view of a humeral head for recreating an anatomical proximate humeral configuration in a shoulder arthroplasty in accord with a second alternative embodiment.

FIG. 4b schematically shows a bottom view of the humeral head of FIG. 4a.

FIG. 5a schematically shows a side view of a humeral head for recreating an anatomical proximate humeral configuration in a shoulder arthroplasty in accord with a third alternative embodiment.

FIG. 5b schematically shows a bottom view of the humeral head of FIG. 5a.

FIG. 5c schematically shows an alternative bottom view of the humeral head of FIG. 5a.

FIG. 5d schematically shows a second side view of the humeral head of FIGS. 5a and 5c, wherein the humeral head is rotated by ninety degrees from the side view shown in FIG. 5a.

FIG. 5e schematically shows an alternative second side view of the humeral head of FIGS. 5a and 5c, wherein the humeral head is rotated by ninety degrees from the side view shown in FIG. 5a.

FIG. 6a schematically shows a perspective view of a cut humeral surface in accord with the preferred embodiment.

FIG. 6b schematically shows mutually axially rotated perspective views of a humerus including the cut humeral surface shown in FIG. 6a.

FIG. 6c schematically shows a perspective view of humeral head coupled to the humeral surface shown in FIGS. 6a and 6b.

FIG. 6d schematically shows an exploded view of the humeral head coupled to the humeral surface shown in a perspective view in FIG. 6c.

FIG. 7a schematically shows a perspective view of a cut humeral surface including a template punch in accord with an alternative embodiment.

FIG. 7b schematically shows mutually axially rotated perspective views of a humerus including the cut humeral surface including the template punch shown in FIG. 7a.

FIG. 8a schematically shows a humerus of a shoulder arthroplasty including a humeral head coupled to a humerus including a cut humeral surface, wherein the shape and size of the periphery of the humeral head matches the anatomic neck and rests on the cortical margins of the humeral surface, in accord with the preferred embodiment.

FIG. 8b schematically shows a rotated view of the humerus of the shoulder arthroplasty of FIG. 8a.

FIG. 8c schematically shows a perspective view of the humerus of the shoulder arthroplasty of FIGS. 8a and 8b.

FIG. 9 illustrates the supero-latero surgical approach in accord with a preferred method.

FIG. 10a schematically illustrates a guide for clamping the humerus for a cutting a humeral head in accord with a preferred embodiment.

FIG. 10b shows the guide of FIG. 10a clamped to a humerus.

FIG. 10c shows how the humeral head is removed from the humerus by a saw that is guided along the surface of the guide clamp of FIG. 10a.

FIGS. 11a-11b illustrate a glenoid version guide in accord with a preferred embodiment.

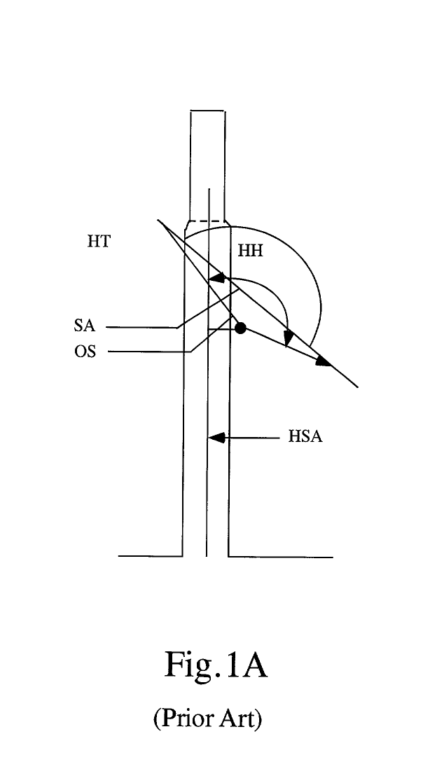

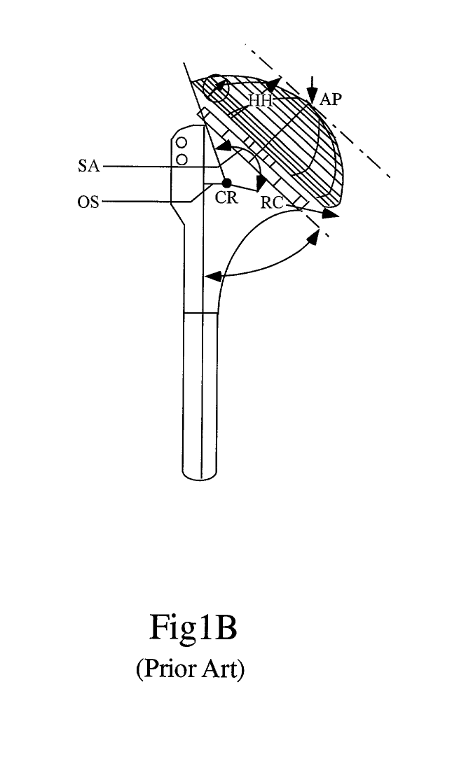

### INCORPORATION BY REFERENCE

What follows is a cite list of references each of which is, in addition to those references cited above in the priority section, hereby incorporated by reference into the detailed description of the preferred embodiment below, as disclosing alternative embodiments of elements or features of the preferred embodiments not otherwise set forth in detail below. A single one or a combination of two or more of these references may be consulted to obtain a variation of the preferred embodiments described in the detailed description below. Further patent, patent application and non-patent references are cited in the written description and are also incorporated by reference into the preferred embodiment with the same effect as just described with respect to the following references:

Boileau P, and Walch G, Mazzolini N, Urien JP. In vitro study of humeral retrotorsion. Journal of Shoulder and Elbow Surgery 2:512, 1993.

Pearl M L, Volk A G. Coronal plane geometry of the proximal humerus relevant to the prosthetic arthroplasty. Journal of Shoulder and Elbow Surgery, 4: 286-289,1995.

Pennington W T, Meyer N J, Zeigler D W. The Glenoid Center Point. An MRI Study of Normal Scapular Anatomy. Medical College of Wisconsin Department of Orthopedic Surgery, Milwaukee, Wis.

Mallon W J, Brown H R, Vogler J B, Martinez S. Radiographic and geometric anatomy of the scapula. Clin. Orthop. 1992, 277: 142-154.

U.S. Pat. Nos.: 4,550,450, 5,507,819, 5,601,562, 5,775,334, 5,895,425, 4,872,451, 4,773,417, 4,901,717, 4,778,473, 5,314,479, 5,779,710, 5,702,486, 5,507,817, 5,330,531, 5,282,865, 5,030,219, 4,919,669, 4,378,607, 3,979,778, 5,906,644, 5,800,551, 5,728,161, 4,279,041, 4,045,826, 3,978,528, 3,694,820, 5,888,203, 5,658,350, 5,597,383, 5,340,362, 4,261,062, and 4,973,211

European Patent Documents No: 0963742 A1, 0940126 A1, 0460886 B1, 0969782 B1 and 0278807 B1.

PCT Applications No.; 99/44546, 98/18412, 97/39693, 98/46172 and 96/17553.

### DETAILED DESCRIPTION OF THE PREFERRED EMBODIMENTS

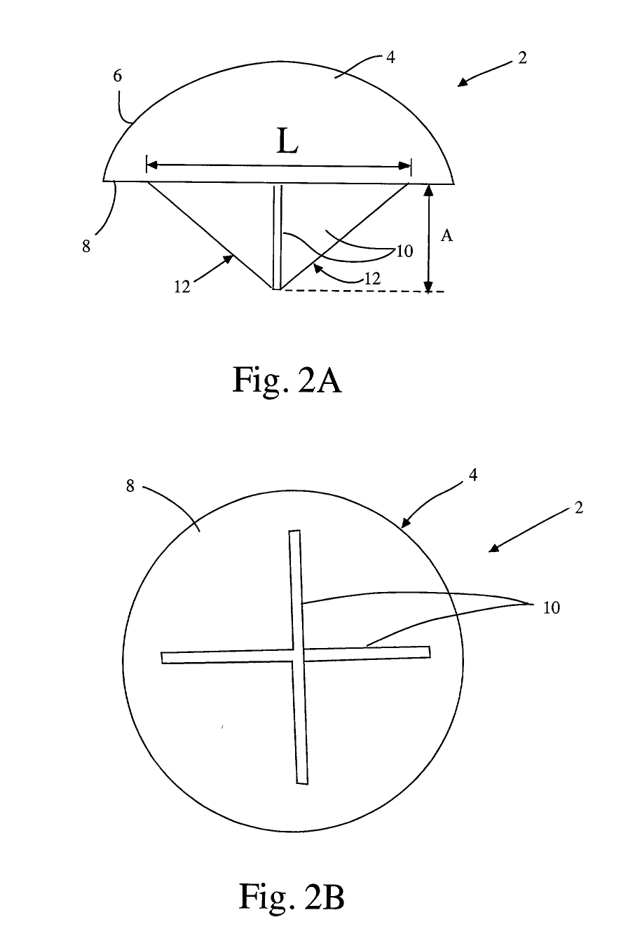

FIG. 2a schematically shows a side view of a humeral head 2 for recreating an anatomical proximate humeral configuration in a shoulder arthroplasty in accord with a preferred embodiment. The humeral head 2 is artificial, preferably comprising a titanium alloy, and is designed to replace a deficient original human humerus that is, e.g., stricken with arthritis or other ailment, or has been injured. The humeral head 2 shown has an upper portion 4 that is formed in the shape of a chord of a sphere, or a cut or partial sphere, i.e., it has a spherical top surface 6 and a circular base 8. The base 8 is a secant of a total sphere. Preferably, the secant has an intersection with a diameter of the sphere, from which the upper portion appears to have been cut, at approximately a distance from the surface 6 to the base 8 which is around one third of the sphere's total diameter.

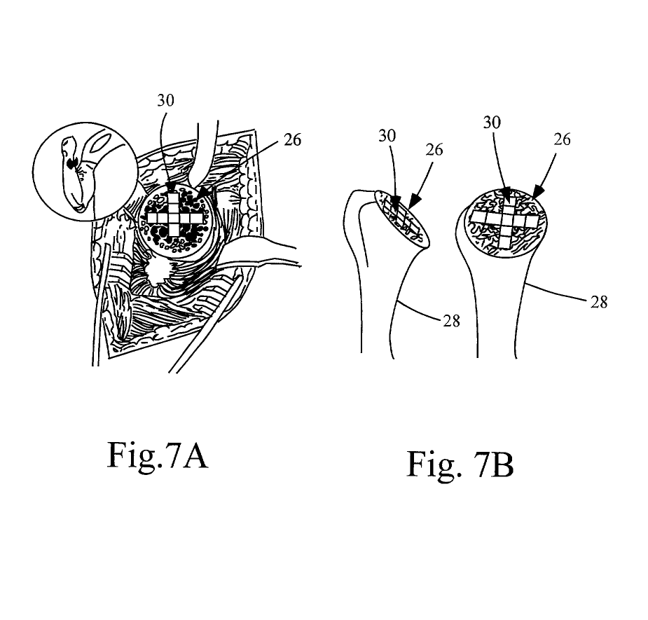

The upper portion 4 of the humeral head 2 may differ from the ideal spherical chord shown in FIG. 2a. As will be discussed below with reference to FIGS. 7a-7c, the shape of the base 8 is preferably formed to substantially match the cortical margins of a cut humeral surface (not shown) to which the humeral head 2 is to be coupled during the arthroplasty. The glenoid to which the humeral head is to be coupled may be an artificial replacement for the original human glenoid (total arthroplasty), or the glenoid may be the original human glenoid (hemiarthroplasty).

A pair of fins 10 according to a preferred embodiment are shown protruding from the base 8 of the humeral head 2. The fins 10 are shown as substantially plano-triangular extensions from the base 8. The two legs 12 forming the vertex of the triangular shape of each fin 10 are preferably straight, as shown, and alternatively may be curved. Also preferably, the triangular fins 10 are substantially isosceles in shape. Each fin 10 may be formed from two right triangular portions wherein the legs 12 are hypotenuses of the right triangles and the right triangular portions forming each fin 10 meet substantially at the center of the other fin 10. As such, the fins 10 intersect.

FIG. 2b schematically shows a bottom view of the humeral head 2 of FIG. 2a. As shown, the preferred fins 10 intersect as described above to form a cruciform shape. The fins 10 may intersect at a right angle or another arbitrary angle such as to form an “X” shape. The bases of the fins 10 may have the same or different lengths L (e.g., L1 and L2, L1≦L2) and the altitudes A may be the same or different (e.g., A1 and A2, A1≦A2), although preferably the lengths L of the bases and altitudes A are the same, as shown.

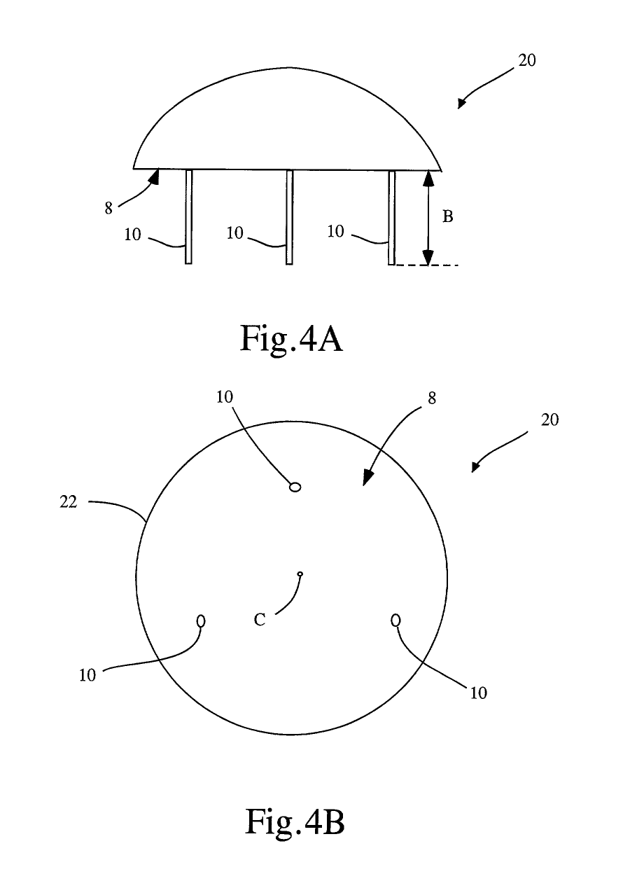

Many other variations of the numbers and shapes of the fins 10 are possible as illustrated below with respect to FIGS. 2a-4e. There may be only one fin 10 and there may be more than two fins 10. The fins 10 need not intersect (see FIG. 4c), and the fins 10 may form a cruciform shape wherein they do not intersect at a vertex, but are rather two isosceles plano-triangular shapes having collinear bases (see FIG. 2c).

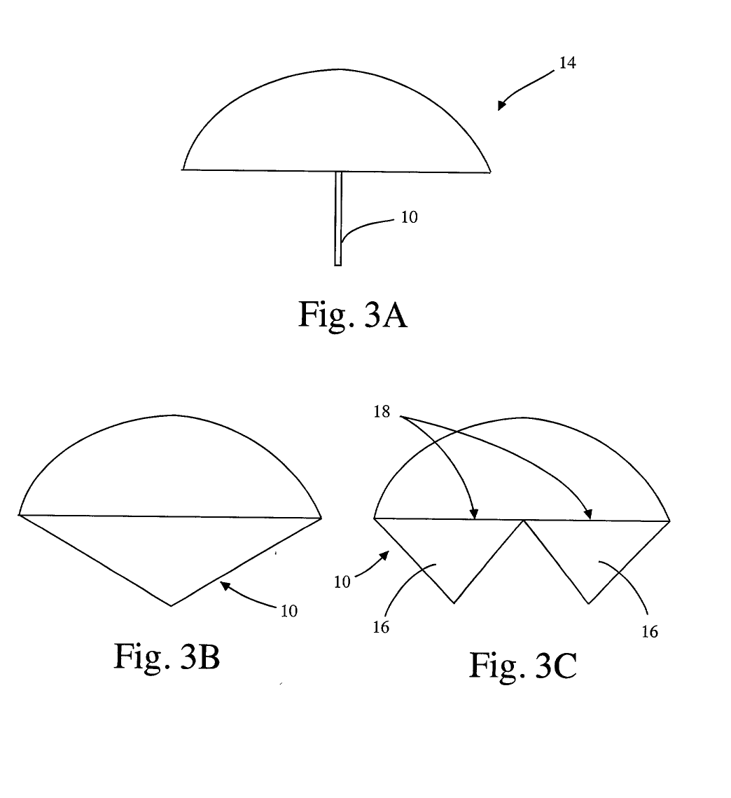

The fin 10 or fins 10 may be arbitrarily plano-polygonal, and they may be linear extensions (see FIGS. 3b and 4b). If the fins 10 are linear extensions, then there will be at least two fins 10 in accord with a preferred embodiment in order that when the fins are coupled to the cancellous tissue of a humeral surface, the fins 10 serve to prevent rotation of the humeral head 2 relative to the humeral surface. Also, the humeral head may include one or more linear fins 10 and one or more planar fins 10 in combination.

FIG. 3a schematically shows a side view of a humeral head 14 for recreating an anatomical proximate humeral configuration in a total shoulder arthroplasty in accord with a first alternative embodiment. The humeral head 14 differs from the humeral head 2 shown in FIGS. 2a-2b in that there is only one fin 10.

FIG. 3b schematically shows a bottom view of the humeral head of FIG. 3a. The fin 10 is shown to be piano-triangular and similar or identical in shape to the fins 10 shown and described with respect to FIGS. 2a and 2b.

FIG. 3c schematically shows an alternative bottom view of the humeral head of FIG. 3a. The fin 10 is shown to be biplano-triangular including two plano-triangular extensions 16 having colinear bases 18. Alternatively, there may be three of more such plano-triangular extensions, or there may be a row of linear extensions. One or both of the fins 10 of the preferred embodiment of FIGS. 2a-2b may have any of the features just mentioned with respect to FIG. 3c.

FIG. 4a schematically shows a side view of a humeral head 20 for recreating an anatomical proximate humeral configuration in a total shoulder arthroplasty in accord with a second alternative embodiment. The humeral head 20 differs from the humeral head 2 shown in FIGS. 2a-2b in that the fins 10 are linear extensions rather than planar extensions. Of course, the linear fins 10 are polyhedral in the sense that they have finite volumetric proportions, as are the planar fins 10 of FIGS. 2a-3c, but the elongated lengths B of the fins 10 of FIGS. 4a-4b are merely larger relative to their extents in the two dimensions transverse to the elongated dimension, than are the altitudes A to the base lengths L of the planar fins 10 of FIGS. 2a-3c. In that sense, the ratios of the sizes of the three dimensions of the fins 10 of the preferred embodiment are intended to be limited only in that the size of the fins 10 in at least one dimension is significantly shorter than that of one of the other dimensions, such that the fins 10 may be “planar” or “linear”.

FIG. 4b schematically shows a bottom view of the humeral head 20 of FIG. 4a. The fins 10 are shown preferably substantially equally spaced from the periphery 22 of the base 8, and are also shown substantially equally spaced from the center C of the base 8. The fins 10 may be otherwise spaced relative to the periphery 22 and the center C. The fins 10 may be spaced at equal or different distances relative to each other. For example, two fins 10 may be closer to each other than each of the two fins 10 are to the third fin 10 (e.g., like the finger holes relative to the thumb hole of a bowling ball).

The small transverse polygonal shapes of the “linear” fins 10 are shown having circular shapes (like needles or pins), but may have any polygonal shape. The transverse dimensions may vary along the lengths B of the fins 10. For example, the fins 10 may taper away from the base 8 beginning an arbitrary distance from the base 8 (like a golf tee, e.g.). The transverse shapes may be the same or different for each of the fins 10. In that sense, the planar fins 10 of FIGS. 2a-3c may also taper away from the base 8 in their short dimension, and need not be rectangular.

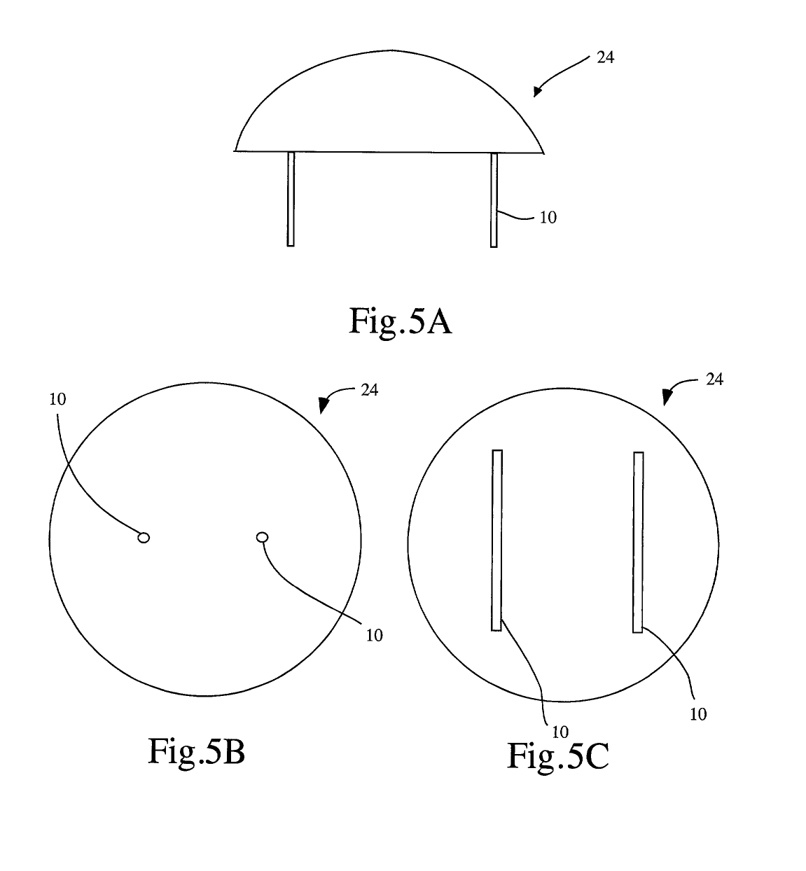

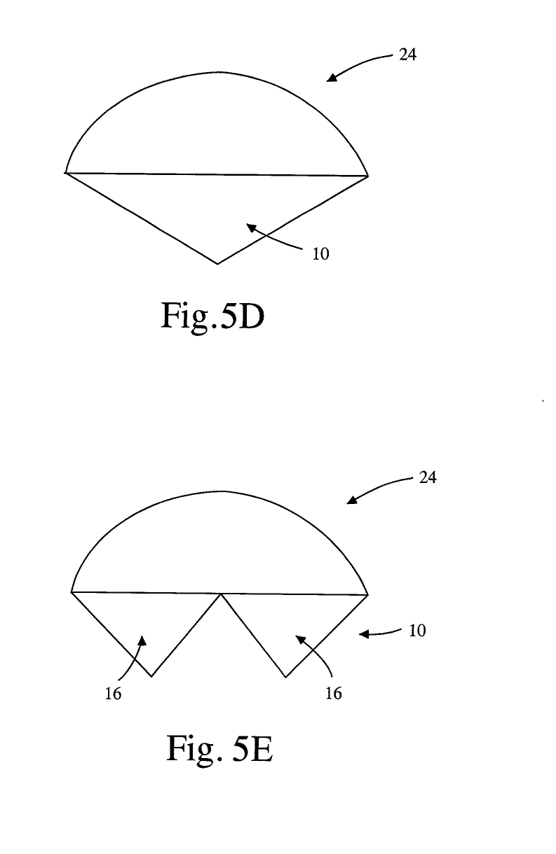

FIG. 5a schematically shows a side view of a humeral head 24 for recreating an anatomical proximate humeral configuration in a total shoulder arthroplasty in accord with a third alternative embodiment. The humeral head 24 differs from the humeral heads 20 and 2, respectively, in that there are only two fins 10 and the fins 10 do not intersect 24.

FIG. 5b schematically shows a bottom view of the humeral head of FIG. 5a. The fins 10 of FIG. 5b are linear as has already been described above with respect to FIGS. 4a and 4b.

FIG. 5c schematically shows an alternative bottom view of the humeral head of FIG. 5a. The fins 10 of FIG. 5c are planar as has already been described above with respect to FIGS. 2a-3c.

FIG. 5d schematically shows a second side view of the humeral head of FIGS. 5a and 5c, wherein the humeral head is rotated by ninety degrees from the side view shown in FIG. 5a. The fins 10 of FIG. 5d are the same or similar to those described above with respect to FIGS. 2a and 3b.

FIG. 5e schematically shows an alternative second side view of the humeral head of FIGS. 5a and 5c, wherein the humeral head is rotated by ninety degrees from the side view shown in FIG. 5a. The fins 10 of FIG. 5e are the same or similar to those described above with respect to FIG. 3c, each fin 10 including two or more planar (and preferably plano-triangular) extensions.

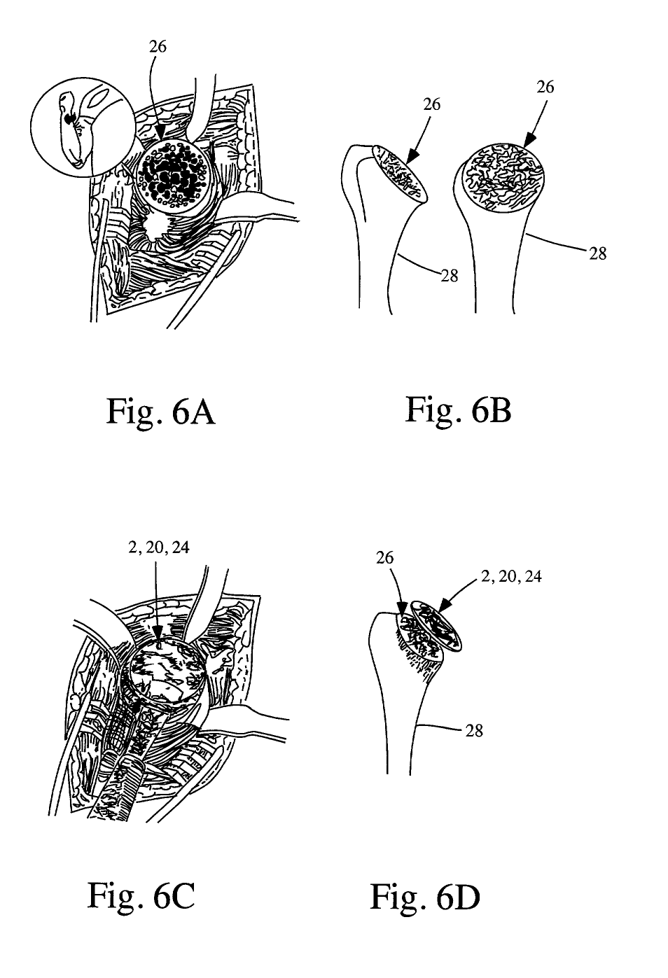

FIG. 6a schematically shows a perspective view of a cut humeral surface in accord with the preferred embodiment. The cut humeral surface is shown as it resides inside of a patient such as during a shoulder arthroplasty operation.

FIG. 6b schematically shows mutually axially rotated perspective views of a humerus 28 including the cut humeral surface shown in FIG. 6a. The humerus 28 shown as been cut in preparation of coupling the humeral head 2, 20 or 24 to the humerus 28 at the humeral surface 26. The cutting preferably results in a substantially planar humeral surface 26 such as would match the base 8 of the humeral head 2, 20, 24. The base of the humeral head 8 could be non-planar, and if so, the cutting would be performed to produce a substantially matching, similarly non-planar humeral surface 26. The humeral surface 26 is preferably cut to expose soft cancellous tissue within which the fins 10 of the humeral head 2, 20, 24 may be impacted. The outer margins of the humeral surface 26 are harder cortical bone tissue.

FIG. 6c schematically shows a perspective view of humeral head 2, 20, 24 coupled to the humeral surface 26 shown in FIGS. 6a and 6b. FIG. 6d schematically shows an exploded view of the humeral head 2, 20, 24 coupled to the humeral surface 26 shown in a perspective view in FIG. 6c. As mentioned above, the humeral head 2, 20, 24 is preferably formed to substantially match the humeral surface 26 both at its periphery and in the contour of its base 8.

FIG. 7a schematically shows a perspective view of a cut humeral surface 26 including a template punch 30 embedded in the humeral surface 26 in accord with an alternative embodiment. FIG. 7b schematically shows mutually axially rotated perspective views of a humerus including the cut humeral surface including the template punch shown in FIG. 7a. The template punch 30 is formed to receive and couple with the fins 10 of the humeral head 2, 20, 24. In that sense, the template punch 30 is a female complement to the protruding fins 10. The template punch 30 shown in FIGS. 7a-7b is the female complement of the cruciform shaped fins 10 shown in FIGS. 2a-2b of the preferred embodiment. The template punch 30 would, however, be differently configured female complements of any of the differently configured alternative embodiments of the fins 10 mentioned above.

The template punch 30 may be a physical apparatus embedded within the cancellous tissue of the humeral surface 26, or merely a preformation of the cancellous tissue to facilitate the coupling of the humeral head 2, 20, 24 with the cut humeral surface 26. In the later preferred case, a template may be placed over the humeral surface 26 and the template punch formed by cutting into the cancellous region beneath and through the slots of the template.

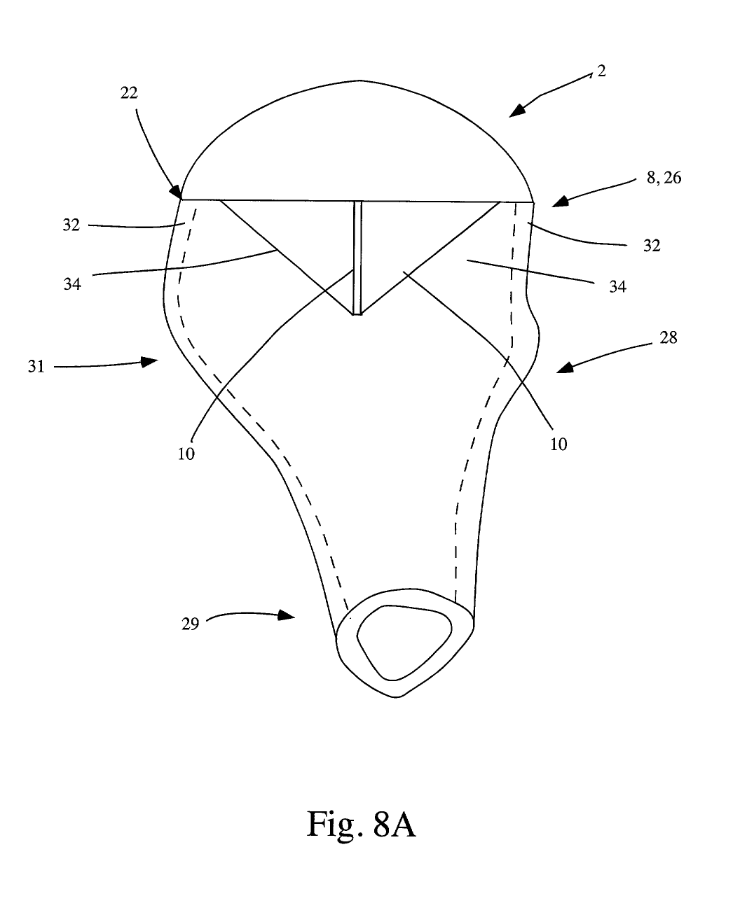

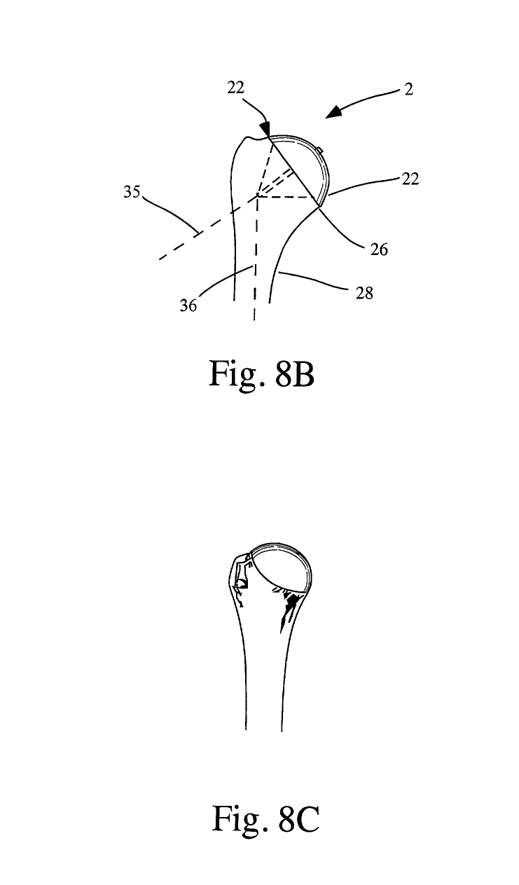

FIG. 8a schematically shows a humeral component of a shoulder arthroplasty including the preferred humeral head 2 of FIGS. 2a-2b coupled to a humerus 28 including a cut humeral surface 26 matched to the base 8 of the humeral head 2. The shape and size of the periphery 22 of the humeral head 2 matches the anatomic neck of the humerus 28 and rests on the cortical margins 32 of the humeral surface 26, in accord with the preferred embodiment. As mentioned above, that periphery 22 may thus be formed to match whatever the polygonal shape of the cortical margins 32 may be for a particular humeral surface 26. The cortical margins 32 are shown at the outer most portion of the humerus 28, and particularly the cut humeral surface 26, whereas the inner portion of the humerus 28 includes the cancellous interior 34. The periphery 22 of the humeral head 2 rests on the hard cortical margins, and as such, is supported there, i.e., from impacting further into the humerus 28.

The fins 10 of the humeral head 2 are shown impacted into the soft cancellous interior 34. The fins 10 may protrude somewhat deeper or less deep than is shown. The stemless design generally means that the base extension or extensions of the humeral head 2, shown as preferably the fins 10 in FIG. 8a, are not protruding down into the elongate region 29 of the humerus 2 and instead are confined within the ball region 31 of the humerus 2. The base extension or extensions shown as the preferred fins 10 in FIG. 8a may insubstantially protrude into the elongate region 29 of the humerus 2, but not in the sense that conventional humeral head replacement devices having stems will protrude substantially down into the elongate region 29 of the humerus 2.

As mentioned above with respect to FIGS. 7a-7b, that cancellous interior 34 may have been preformed to receive the fins 10 via a template punch 30. Preferably, the fins 10 are adhesively coupled to the cancellous interior 34 using, e.g., surgical cement such as methylmethacrylate. Alternatively, the fins 10 are press-fit to the cancellous tissue 34 of the humeral surface 26.

FIG. 8b schematically shows a rotated view of the humeral component of the shoulder arthroplasty of FIG. 8a. FIG. 8b illustrates an advantage of the preferred embodiments which is that the humeral head 2 is stemless. The normal 35 to the humeral surface 26 is shown rotated from the elongated axis 36 of the humerus 28 by about 45°, and may be rotated by different angles typically significantly deviated from colinear. This makes it difficult to insert or remove a conventional humeral head having a long stem (e.g., six inches or so in length). In contrast, the humeral head 2 of the preferred embodiment may be inserted without complications caused by the non-colinearity of the humeral surface normal 35 and the elongate humeral axis 36 (the fins 10 having, e.g., 3/4 inch altitudes or lengths, and being, e.g., less than two inches wide at their bases).

In addition, since the humerus 28 can be differently formed, particularly in this elongated dimension, from patient to patient, each conventional humeral head has a stem that is uniquely and precisely formed for each patient. This greatly increases the complexity and expense of the arthroplasty, as well as any revision of the arthroplasty that may later be performed. In contrast, the fins 10 of the humeral head 2 of the preferred embodiment may be similarly or identically shaped for most if not all configurations of the humerus 28. Only the periphery 22 of the humeral head 2 is preferably differently formed (e.g., varied between 25 mm and 60 mm in diameter) depending on the size and shape of the humeral surface 26 of the humerus 28 of the particular patient. FIG. 8c schematically shows a perspective view of the total shoulder arthroplasty of FIGS. 8a and 8b.

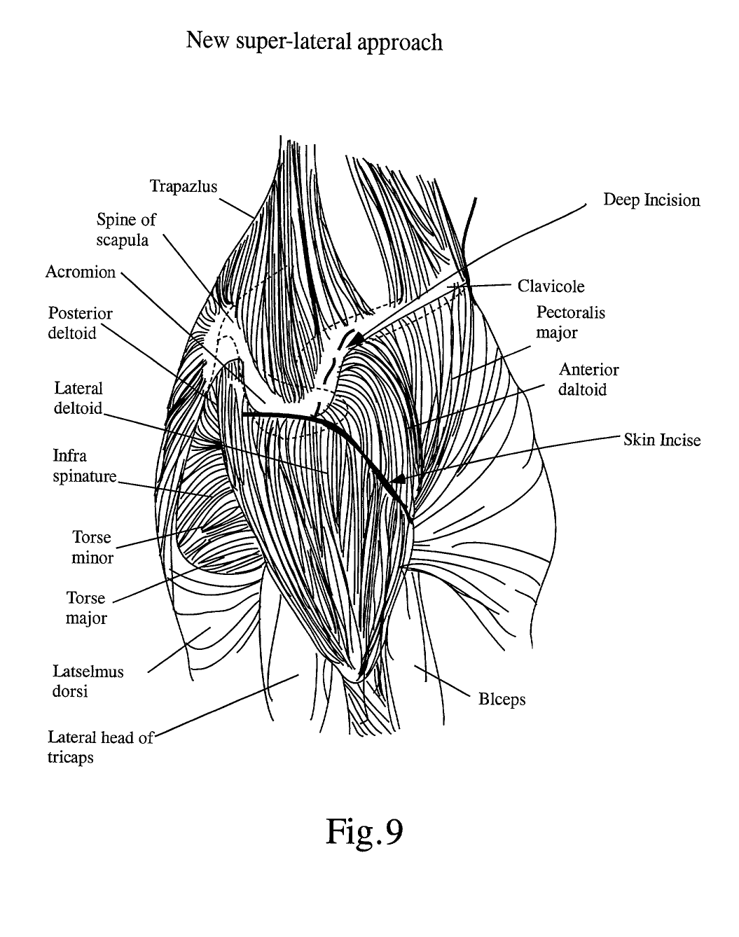

The surgical approach is a supero-lateral deltoid splitting approach (see FIG. 9), as opposed to the delto-pectoral approach used in all or most other systems. This approach will allow much improved access to the glenoid. The skin incision is from the postero-lateral corner of the acromion to the axillary crease. After subcutaneous undermining the deep layer incision begins on the superior surface of the clavicle, continues over the acromioclavicular joint and anterior margin of the acromion. The deltoid is split to the extent of the subacromial bursa. The axillary nerve is palpated and protected during the procedure. The acromio-clavicular capsule and coraco-acromial ligament is dissected from their bony attachments and tagged to insure their restoration on closing. Three centimeters of anterior deltoid medial to the acromio-clavicular joint is dissected subperiosteally from the clavicle. This will create a large anterolateral access to the glenohumeral joint. The glenohumeral joint is then opened in the usual fashion by cutting through the subscapularis and capsule. This approach avoids easier and direct glenoid access without posterior capsular releases and vigorous retraction.

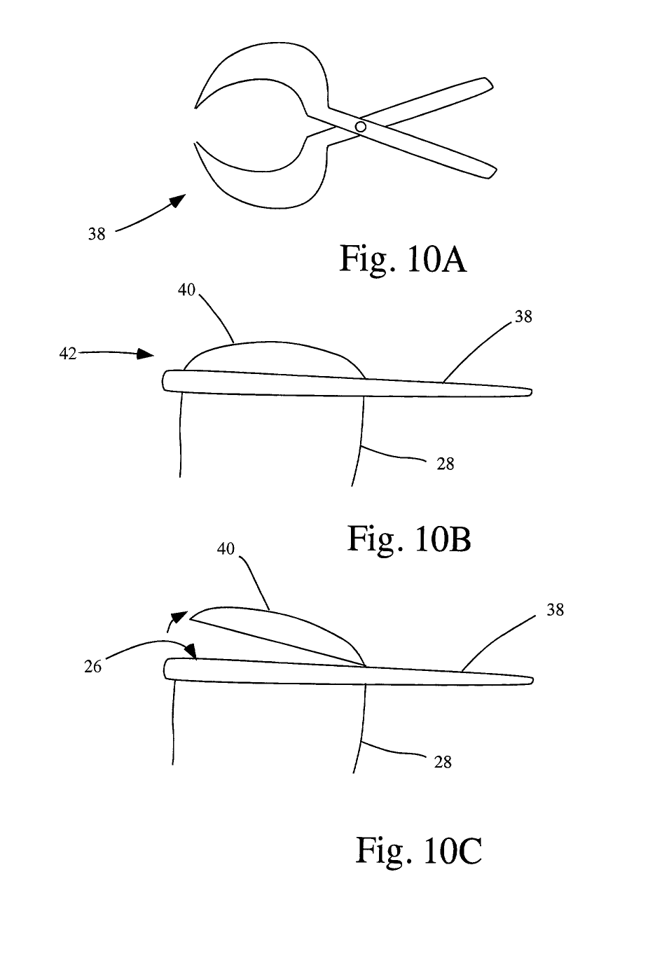

The method includes a cutting jig to guide an oscillating saw to cut at the anatomic neck of the humerus. There will be right and left shoulder guide for this purpose. The guide will be similar to a large tenaculum and will open and close onto the anatomic neck. It will have a variable axis as a typical pair of pliers. It will encompass preferably less than a full 360 degrees, and particularly preferably only 240 degrees, of the circumference of the anatomical neck. The guide will protect the rotator cuff and make use of the bald non-articular area posteriorly using an 8 mm thickness posteriorly to correctly cut on the anatomic neck. (see FIGS. 10a-10c).

FIG. 10a shows a schematic of the guide 38 to be used for guiding the saw to cut the humerus to reveal the humeral surface 26. FIG. 10b shows the guide 38 as it is clamped to the humerus 28, wherein the original anatomical humeral head 40 is held above the surface 42 of the guide 38. A saw is then used to cut along the surface 42 and remove the original head 40 from the humerus 28, as illustrated at FIG. 10c. The cut humeral surface 26 mentioned above is thus revealed with precision using the guide 38 of the preferred embodiment.

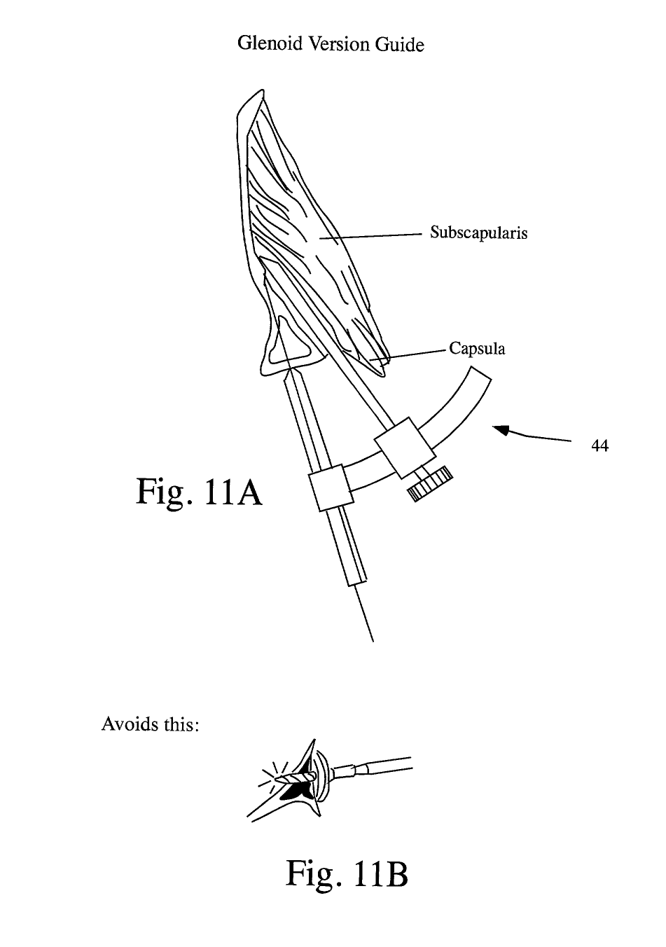

The glenoid version guide 44 is another advantageous tool in cases of excessive posterior wear or any type of glenoid destruction that creates alignment (version) problems. (See FIGS. 11a-11b).

### Object of the Invention Met

“The Facile Shoulder” meets the above object of the invention, and solves several problems associated with conventional total shoulder arthroplasty (TSA), some of which have been referred to above. Some of the advantages, not already discussed above with respect to the preferred and alternative embodiments including those specifically shown in FIGS. 2a-8c, of the facile total shoulder arthroplasty of the preferred embodiments are described below.

### Recreating anatomic proximal humeral configuration with a “stemless humeral component”.

This is achieved in accordance with the facile total shoulder arthroplasty of the preferred embodiments, i.e. “a contoured surface replacement seated on a periphery of cortical bone with stabilizing intraosseus extensions into the cancerous bone.” The efforts made in development of TSA have often been patterned after total hip arthroplasty (THA), but the shoulder is not a constrained ball and socket joint, nor is it a weight bearing joint in the true sense of the term. In fact, the shoulder is more akin to the knee joint than it is to the hip joint. For example, the stability of both TSA and TKA is based on the integrity of the soft tissues (ligamentous and muscular) supporting these joints. The tibial components of primary total knee arthroplasty utilize components that are cemented onto contoured surfaces of the femur and tibia. In spite of the significant forces exerted on TKA components by the weight-bearing functions of the knee joint, there are no stem-like extensions that go into the tibia or femur in primary total knee arthroplasty. The fixation of the components in TKA is based on the proper fit on contoured cancellous and peripheral cortical surfaces.

### The Facile Humeral head is stemless

The anatomic heads (⅓ spheres from 36 to 52 mm in diameter) has triflanged extension that provides stability and fixation into the cut cancellous surface, while the periphery of the head rests on the cortical margins and calcar of the anatomic neck. The components could be designed to be cemented or press-fit, but the initial series are preferably of the cemented design.

The Facile shoulder is an extremely cost effective TSA. The cemented design with an all polyethylene glenoid will be most cost effective TSA in the marketplace.

Accurate anatomic positioning of the humeral head without the need for a complex and expensive humeral stem will be a great advantage. The Facile Shoulder's stemless design makes it easy to adapt perfectly to the patient's angle of inclination, retroversion and medial offset since it does not have to relate to a stem. Other “anatomic” TSA systems have stems that have to coapt with the humeral head. It is difficult to obtain an accurate match with the angle and medial offset of the cut surface of the proximal humerus in a “stemmed system” The rotation around an offset Morse taper frequently produces significant compromises in the coverage and matching of the head component and the cut surface.

In the Facile Shoulder the proper size template is chosen to accurately cover the cut cortical/cancellous surface. It is easily positioned and a triflanged cut is made through the slots in the template into the cancellous surface. Third generation cementing techniques are used to fixate the component on the humerus. The prosthesis is a simple one-piece component that should be extremely price competitive.

The anatomic neck cutting guide is an advantageous instrument in this technique. There are believed to be no similar existing guides for any TSA system. It will produce an accurate and consistent cut while protecting the rotator cuff. The glenoid version guide is unique and is based on solid anatomic and scapular image analysis.

The Facile Shoulder is designed for easy revision. The amount of bone affected by revision would be minimal due to its stemless design. It would be easy to revise it to a stemmed design at a later date. The prosthesis is designed for easy removal with a groove just under the peripheral edge.

Those skilled in the art will appreciate that the just-disclosed preferred embodiments are subject to numerous adaptations and modifications without departing from the scope and spirit of the invention. Therefore, it is to be understood that, within the scope and spirit of the invention, the invention may be practiced other than as specifically described above. The scope of the invention is thus not limited by the particular embodiments described above. Instead, the scope of the present invention is understood to be encompassed by the language of the claims that follow, and structural and functional equivalents thereof.

In addition, in the method claims that follow, the steps have been ordered in selected typographical sequences. However, the sequences have been selected and so ordered for typographical convenience and are not intended to imply any particular order for performing the steps.

I claim:

1. A total shoulder arthroplasty apparatus for recreating an anatomic proximal humeral configuration, comprising:
a stemless humeral head for coupling to a cut humeral surface, wherein the humeral head includes a base having a stabilizing base extension protruding therefrom for impaction into a cancellous region of the cut humeral surface.

2. The apparatus of claim 1, wherein the protruding base extension includes one or more fins for rotational stabilization.

3. The apparatus of claim 2, wherein the one or more fins are substantially planar.

4. The apparatus of claim 1, wherein the protruding base extension includes at least two fins formed to have a cruciform shape.

5. The apparatus of any of claims 1, wherein the shape of the one or more fins is plano-triangular.

6. The apparatus of claim 1, wherein the protruding base extension further includes at least one linear fin.

7. The apparatus of claim 1, wherein the cancellous region said base extension protrudes into is non-intramedullary.

8. A total shoulder arthroplasty apparatus for recreating an anatomic proximal humeral configuration, comprising:
a stemless humeral head for coupling to a previously cut humeral surface, wherein the humeral head includes a base having a non stem-bearing stabilizing base extension protruding therefrom for impaction into a cancellous region of the cut humeral surface.

9. The apparatus of claim 8, wherein the protruding base extension includes two or more linear extensions for rotational stabilization.

10. The apparatus of any of claims 1 or 8, wherein the periphery of the humeral head is formed to match cortical margins of the cut humeral surface.

11. The apparatus of any of claims 1 or 8, wherein the humeral head is attached to the humeral surface using an adhesive.

12. The apparatus of claim 11, wherein the adhesive is surgical cement.

13. The apparatus of any of claims 1 or 8, wherein the humeral head is attached to the humeral surface by press-fitting.

14. The apparatus of any of claims 1 or 8, wherein the periphery of the base of the humeral head is formed to match a specific shape and size of the anatomic neck of a specific humeral surface.

15. The apparatus of any of claims 1 or 8, further comprising a template punch inserted into the cut humeral surface, wherein the base extension is a total or partial male complement to the female template punch.

16. A total shoulder arthroplasty apparatus for recreating an anatomic proximal humeral configuration, comprising:
a stemless humeral head for coupling to a cut humeral surface, wherein the humeral head includes a base having a stabilizing base extension protruding therefrom for impaction into a cancellous, non-intramedullary region of the cut humeral surface.

17. The apparatus of claim 16, wherein the periphery of the humeral head is formed to match cortical margins of the cut humeral surface.

18. The apparatus of claim 16, wherein the humeral head is attached to the humeral surface using an adhesive.

19. The apparatus of claim 18, wherein the adhesive is surgical cement.

20. The apparatus of claim 16, wherein the humeral head is attached to the humeral surface by press-fitting.

21. The apparatus of claim 16, wherein the periphery of the base of the humeral head is formed to match a specific shape and size of the anatomic neck of a specific humeral surface.

22. The apparatus of claim 16, further comprising a template punch inserted into the cut humeral surface, wherein the base extension is a total or partial male complement to the female template punch.

23. A total shoulder arthroplasty apparatus for recreating an anatomic proximal humeral configuration, comprising:
a humeral head for coupling to a cut humeral surface, wherein the humeral head includes a base having a stabilizing base extension protruding therefrom for impaction into a cancellous region of the cut humeral surface, and wherein the base extension is confined to protrude only into a ball region of the humerus, to which the humeral head couples, and which is above an elongate region of the humerus.

24. A total shoulder arthroplasty apparatus for recreating an anatomic proximal humeral configuration, comprising:
a humeral head for coupling to a cut humeral surface, wherein the humeral head includes a base having a stabilizing base extension protruding therefrom for impaction into a cancellous region of the cut humeral surface, and wherein the extension is nonintrusive of an elongate humeral region below a humeral ball region including the humeral head.

25. A total shoulder arthroplasty method for recreating an anatomic proximal humeral configuration, comprising the steps of:
preparing a stemless humeral head having a base including a stabilizing base extension for efficient rotational stabilization of the humeral head on a cut humeral surface for coupling with the cut humeral surface;
preparing a humeral surface for coupling the humeral head thereto, including cutting the humeral surface to reveal a cancellous interior; and
coupling the humeral head to the humeral surface, thereby recreating the anatomic proximal humeral configuration.

26. The method of claim 25, wherein said stabilizing base extension includes one or more fins.

27. The method of claim 26, wherein said one or more fins are substantially planar in shape.

28. The method of claim 27, wherein the planar shape of the one or more fins is triangular.

28. The method of claim 25, wherein the stabilizing base extension includes two fins formed to have a cruciform shape.

30. The method of claim 25, wherein the protruding base extension further includes at least one linear fin.

31. A total shoulder arthroplasty method for recreating an anatomic proximal humeral configuration, comprising the steps of:
preparing a stemless humeral head having a base including a non stem-bearing stabilizing base extension for rotational stabilization of the humeral head on a cut humeral surface for coupling to the cut humeral surface;
preparing a humeral surface for coupling the humeral head thereto, including cutting the humeral surface to reveal a cancellous interior; and
coupling the humeral head to the humeral surface, thereby recreating the anatomic proximal humeral configuration.

32. The method of claim 31, wherein the base extension includes two or more linear extensions.

33. The method of any of claims 25 or 31, wherein the coupling step includes the step of impacting the base extension into the cancellous of the cut humeral surface, wherein the periphery of the humeral head rests on the cortical margins of the humeral surface following the coupling.

34. The method of any of claims 25 or 31, wherein the coupling step includes the step of adhesively coupling the humeral head to the humeral surface.

35. The method of claim 34, wherein the adhesive is surgical cement.

36. The method of any of claims 25 or 31, wherein the coupling step includes the step of press-fitting the humeral head to the humeral surface.

37. The method of any of claims 25 or 31, wherein the forming step includes the step of selecting a shape and size of the periphery of the base of the humeral head from a variety of shapes and sizes for matching the specific shape and size of the anatomic neck of the cut humeral surface.

38. The method of any of claims 25 or 31, further comprising the step of inserting a template punch into the cancellous of the cut humeral surface prior to performing the coupling step.

39. The method of claim 38, wherein the base extension is a total or partial male complement to the female template punch.

40. The method of any of claims 25 and 31, wherein the coupling step includes impacting the base extension of the humeral head to protrude only into a ball region of the humerus above an elongate region of the humerus.

41. The method of any of claims 25 and 31, wherein the coupling step includes impacting the base extension of the humeral head nonintrusive to an elongate region of the humerus below a ball region of the humerus.

42. A total shoulder arthroplasty method for recreating an anatomic proximal humeral configuration, comprising the steps of:
preparing a stemless humeral head having a base including a non stem-bearing stabilizing base extension for rotational stabilization of the humeral head on a cut humeral surface for coupling to the cut humeral surface;
preparing a humeral surface for coupling the humeral head thereto, including cutting the humeral surface to reveal a cancellous interior; and
coupling the humeral head to the humeral surface, thereby recreating the anatomic proximal humeral configuration, including impacting the base extension of the humeral head to protrude only into a ball region of the humerus above an elongate region of the humerus.

43. A total shoulder arthroplasty method for recreating an anatomic proximal humeral configuration, comprising the steps of:
preparing a stemless humeral head having a base including a non stem-bearing stabilizing base extension for rotational stabilization of the humeral head on a cut humeral surface for coupling to the cut humeral surface;
preparing a humeral surface for coupling the humeral head thereto, including cutting the humeral surface to reveal a cancellous interior; and
coupling the humeral head to the humeral surface, thereby recreating the anatomic proximal humeral configuration, including impacting the base extension of the humeral head nonintrusive to an elongate region of the humerus below a ball region of the humerus.

44. The method of any of claims 25, 31 or 42-43 wherein said preparing step comprises the steps of:
surgically establishing an access to a humerus of a patient;
coupling a guide to the humerus, wherein the humeral head remains exposed;
positioning said guide to define a humeral surface; and
removing said humeral head by cutting along said humeral surface defined by said guide, whereby a precise humeral surface is revealed for attaching an artifical humeral head during said arthroplasty.

45. The method of claim 44, further comprising the step of aligning said humeral surface with a glenoid version guide.

46. The method of claim 44, further comprising the steps of:
preparing for coupling to the cut humeral surface a stemless humeral head having a base including a non stem-bearing stabilizing base extension for rotational stabilization of the humeral head on a cut humeral surface; and
coupling the humeral head to the humeral surface, thereby recreating the anatomic proximal humeral configuration.

47. A method for performing a shoulder arthroplasty, comprising the steps of:
surgically establishing an access to a humerus of a patient;
coupling a guide to the humerus, wherein the humeral head remains exposed;
positioning said guide to define a humeral surface; and
removing said humeral head by cutting along said humeral surface defined by said guide, whereby a precise humeral surface is revealed for attaching an artifical humeral head during said arthroplasty.

48. The method of claim 47, further comprising the step of aligning said humeral surface with a glenoid version guide.

49. The method of claim 47, further comprising the steps of:
preparing for coupling to the cut humeral surface a stemless humeral head having a base including a non stem-bearing stabilizing base extension for rotational stabilization of the humeral head on a cut humeral surface; and
coupling the humeral head to the humeral surface, thereby recreating the anatomic proximal humeral configuration.

50. A total shoulder arthroplasty method for recreating an anatomic proximal humeral configuration, comprising the steps of:
preparing a stemless humeral head having a base including a non stem-bearing stabilizing base extension for rotational stabilization of the humeral head on a cut humeral surface for coupling to the cut humeral surface;
preparing a humeral surface for coupling the humeral head thereto, including cutting the humeral surface to reveal a cancellous interior; and
coupling the humeral head to the humeral surface, thereby recreating the anatomic proximal humeral configuration.
